(19) United States
(12) Patent Application Publication
(10) Pub. No.: US 2018/0043042 A1
(43) Pub. Date: Feb. 15, 2018

(54) METHOD AND TREATMENT STATION FOR HEATING AND STERILIZED KEGS IN PARTICULAR REUSEABLE KEGS

(71) Applicant: KHS GmbH, Dortmund (DE)

(72) Inventor: Hilmar FICKERT, Kriftel (DE)

(21) Appl. No.: 15/552,378

(22) PCT Filed: Feb. 3, 2016
(86) PCT No.: PCT/EP2016/052258

§ 371 Date: Aug. 21, 2017

(30) Foreign Application Priority Data
Feb. 20, 2015 (DE) 10 2015 102 401.2

(51) Int. Cl. A61L 2/07; B65B 55/10

(52) U.S. Cl. CPC A61L 2/07; B65B 55/10; A61L 2202/23; A61L 2202/17; A61L 2202/15

(57) ABSTRACT

A pressurized phase for vapor treating a reusable metal keg includes filling the keg with pressurized water vapor through an inlet and then interrupting the connection, thus initiating a dwell time. During this dwell time, condensate forms on the inner surfaces of the keg. Meanwhile, one causes relative movement between the water vapor and the keg's inner walls.

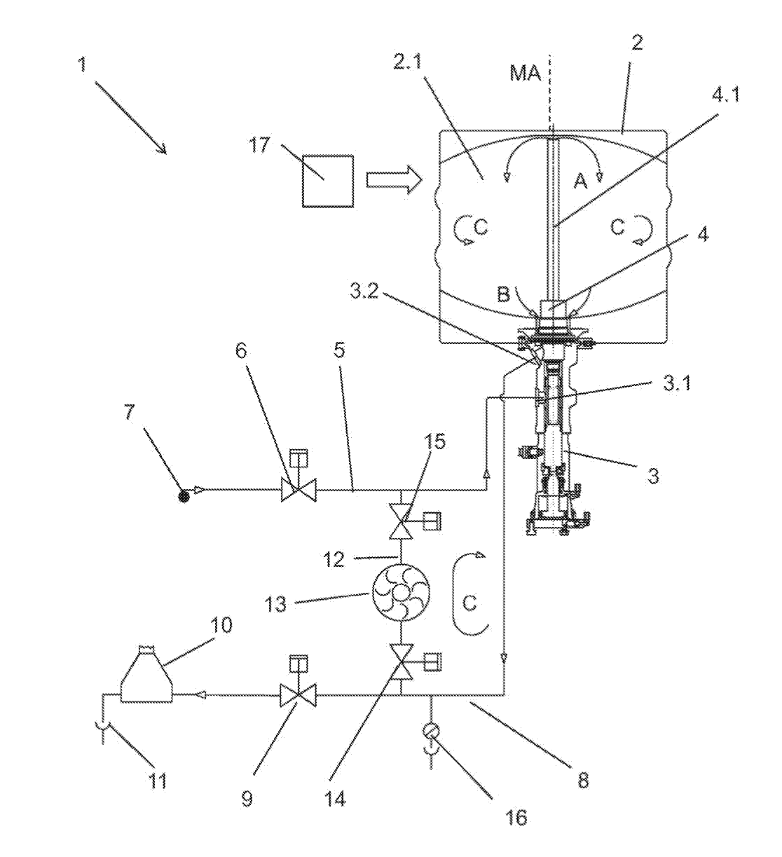

### RELATED APPLICATIONS

This is the national stage of PCT/EP2016/052258, filed on Feb. 3, 2016, which claims the benefit of the Feb. 20, 2015 priority date of German application DE 102015102401.2

### FIELD OF INVENTION

The invention relates to reusable kegs, and in particular, to treatment of such kegs so that they can be reused.

### BACKGROUND

Reusable kegs are known. The kegs are filled with some liquid filling, such as beer, wine, juice, and/or water. After the product has been consumed, the kegs, which are now empty, are returned for reuse.

Preparing a keg for reuse includes treating the keg by emptying residues and cleaning it. After the keg has dispensed its contents, it is typically returned for cleaning and refilling.

Near the end of the cleaning process, it is usual to expose the keg's interior to a sterile gas or vapor and to sterilize the interior by heating the interior with water vapor before refilling.

It is important to achieve rapid and uniform heating of all areas of the keg's inner surface, including its keg fitting for the required time. The temperature should be high enough to sterilize and should be maintained for an extended period and as uniformly as possible. However, because of the sealing material used in a keg fitting, which is typically EPDM, the temperature should not exceed 135° C. for an extended period.

After the above treatment, it is typical to pre-stress the keg interior with an inert gas such as carbon dioxide gas. This also flushes out any oxygen.

A known way to carry out vapor treatment is to have a pressureless phase followed by a pressure phase.

The pressureless phase includes blowing water vapor through the keg's interior for several seconds so that it passes from an inlet and to an outlet, taking any condensation with it. During this period, the keg is heated slightly, but not to an extent that would promote sterilization.

In the pressure phase that follows, the outlet closes, thus allowing pressure of incoming water vapor to increase. At some value of pressure, the inlet is also closed and the keg is permitted to sit for a suitable dwell time. During this dwell time, heat from the water vapor transfers to the container wall. This causes water to condense. As it does so, the pressure in the keg's interior decreases. This dwell time typically lasts sixty to ninety seconds. Following this dwell time, an inert gas purges the keg's interior.

### SUMMARY

The invention is based in part on the recognition that the pressureless phase consumes a great deal of energy without significantly heating the keg's interior and that, during the pressure phase, the rapid condensation that occurs on the wall and that collects on the keg's bottom or elsewhere interferes with heat transfer onto those surfaces.

The preferred area of application of the invention is the heating and sterilization of reusable kegs. The invention also applies, however, to kegs in general, and to the first use of kegs that have not previously been used or reusable kegs, i.e. before the first filling of these not-yet-used kegs or reusable kegs. The vapor treatment takes place at least with reusable kegs after a keg-interior cleaning.

By intensifying relative movement between the water vapor and the inner surfaces of the treated keg, it is possible to achieve more substantial condensation and heat transfer on all the regions of the keg inner surfaces and surfaces of the keg fitting.

The foregoing intensification also shortens of pressureless vapor treatment phase. This is particularly advantageous because this is the phase that incurs the higher energy consumption. Also as a result of the intensification, the pressure phase of the vapor treatment becomes substantially faster than it is in known methods.

With the relative movement between the water vapor and the keg inner surfaces, the removal of the condensation formed there is achieved in particular, and therefore the situation is achieved that the condensation which has formed at a region of the keg inner surface prevents water vapor from condensing again at this region, and thereby contributes to the heating of the keg. Rather, due to the intensive relative movement, condensation is caused to run off or drain away, such that at all the regions water vapor can condense and thereby directly allow for a further heating up of the keg inner surface.

Due to the shortening of the pressureless phase, and also due to the shortening of the holding time or leave-in time, in overall terms without impairment of the heating striven for of the respective keg, and therefore without impairment of the quality of the sterilization of the respective keg, a reduction is also achieve of the consumption of water vapor and energy, and a reduction of the treatment duration necessary for the heating. The latter consideration therefore also allows for a substantial increase in the performance capacity of a machine or system for the treatment and, respectively, for the cleaning, sterilizing, and filling of kegs, i.e. an increase in the number of kegs treated per time unit.

The relative movement between the water vapor and the inner surfaces of the keg interior can be produced in various ways, for example in that, at least during the leave-in or holding time, i.e. with the keg interior separated from the water vapor delivery, the water vapor which is presented there is circulated, specifically by the leading off of the water vapor from the keg interior and then the reintroduction of the water vapor into the keg interior, in a closed circuit.

Moreover, the relative movement between the water vapor in the keg interior and the inner surfaces of the keg can also be produced mechanically, for example by a movement of the keg, e.g. by rotation, by jerking or shaking, for example by repeated acceleration and deceleration of the keg, etc. In principle, this mechanical movement of the keg can also be combined with the circulation of the water vapor that is causing the relative movement.

The expression “essentially” or “approximately” signifies in the meaning of the invention deviations from the exact value in each case by +/−10%, preferably by +/−5%, and/or deviations in the form of changes which are not of significance for the function.

Further embodiments, advantages, and possible applications of the invention are also derived from the following description of exemplary embodiments and from the Figures. In this context, all the features described and/or pictorially represented are in principle objects of the invention, individually or in any desired combination, regardless of their association in the claims or reference to them. The contents of the claims are also deemed constituent parts of the description.

The invention is described in detail hereinafter in connection with the figures and in relation to an exemplary embodiment.

### DESCRIPTION OF THE FIGURE

The FIGURE shows a schematic diagram of a treatment position of a processing machine.

### DETAILED DESCRIPTION

The FIGURE shows a treatment position 1 of a machine or system for emptying, cleaning, sterilizing, and subsequent filling of a keg 2 having a keg fitting 4 that includes a riser pipe 4.1. As used herein, the term “keg” refers to reusable kegs or multiple-use kegs, in particular metallic reusable kegs that are often filled with a liquid filling product, for example with beverages such as beer, wine, juice, or water. Details of the machine that are not essential to understanding the invention are omitted for clarity.

The FIGURE shows the keg 2 that is oriented upside-down with its keg fitting 4 pointing downwards and sealed against a treatment head 3. Also shown in the FIGURE are certain function elements of the treatment position 1 that are necessary for heating the keg 2, and in particular, its wall or keg fitting 4. Among these function elements are a first line 5, a second line 8, and a bypass line 12 that extends between the first and second lines 5, 8.

Along the first line 5 is a first control-valve 6 that connects a water-vapor inlet 7 with a treatment-head inlet 3.1 of the treatment head 3. Similarly, along the second line 8 is a second control-valve 9 that connects a treatment-head outlet 3.2 of the treatment head 3 with a liquid separator 10. One example of a liquid separator 10 is a cyclone.

The liquid separator 10 assists in separating condensate that collects as a result of condensation that occurs in a pressureless phase of the keg treatment. In the illustrated embodiment, the second line 8 carries this separated condensate into a gully 11.

A third control-valve 14, a pump 13, and a fourth control-valve 15 extend in series along the bypass line 12. The pump 13 has an inlet that connects to the third control-valve 14 and an outlet that connects to the fourth control-valve 15. A typical pump 13 is a circulating pump, a vapor pump, a blower or a fan.

The flow from the water-vapor inlet 7 to the treatment-head inlet 3.1 defines a first flow direction. The bypass line 12 connects downstream of the first control-valve 6 in this flow direction. Similarly, the flow from the treatment-head outlet 3.2 to the separator 10 defines a second flow direction. The bypass line 12 connects to the second line 8 upstream of the second control-valve 9 relative to this second flow direction

During the pressureless phase of the keg treatment, the treatment-head inlet 3.1 places the first line 5 in communication with the riser pipe 4.1 so that water vapor emerges at the top of the riser pipe 4.1 into the keg's interior 2.1 and flows in a first flow direction A. In addition, the treatment-head outlet 3.2 opens into the second line 8. This means that water vapor and condensed water can be conveyed away along a second flow direction B.

The FIGURE shows only those function elements that are necessary for the vapor treatment. It does not show function elements that are required for other treatment steps and that are, for those purposes, likewise in connection with the treatment head 3, such as, for example, those function elements that are necessary for the interior cleaning of the reusable keg 2 with different cleaning or treatment media, and for the removal or expulsion of the last cleaning medium at the end of the interior cleaning, especially since it is in principle also possible for the interior cleaning of the reusable keg and the subsequent heating and sterilizing of this keg to be carried out by vapor treatment at different treatment heads.

Heating and sterilizing the keg 2 that is sealed against the treatment head 3 takes place after the keg's interior 2.1 has been cleaned and after all treatment media have been expelled. With the third and fourth control-valves 14, 15 closed and the water vapor pump 13 not activated, but with the first and second control-valves 6, 9 opened, and with the inlet and return of the treatment head 3 and of the fitting 4 open, water vapor briefly blows through the keg's interior 2.1.

In this pressureless phase, the water vapor is delivered under pressure from the water-vapor inlet 7 at a temperature of not more than 135° C. The water vapor proceeds via the first line 5 and the opened first control-valve 6 to the treatment-head inlet 3.1. It then emerges at the upper end of the riser pipe 4.1 along a first flow direction A. After flowing through the interior 2.1, the water vapor is conveyed, together with any condensate to the separator 10 via the treatment-head outlet 3.2 and the opened second line 8. The separator 10 separates the vapor and condensate and directs the latter to the gully 11. During this pressureless phase, some heating and sterilization already takes place on the inner surfaces of the keg 2 and of the keg fitting 4 as water vapor condenses.

The pressure phase of the vapor treatment starts with blocking the returns of the treatment head 3 and of the keg fitting 4. Since the treatment head inlet 3.1 and the keg fitting 4 continue to be connected to the water-vapor inlet 7 via the opened first line 5, pressure of water vapor will build in the keg interior 2.1. At some point, which is determined either by lapse of time or achievement of some pressure, the first control-valve 6 closes and thus blocks the first line 5. This separates the water-vapor inlet 7 from the interior 2.1. The second control-valve 9 remains closed. The third and fourth control-valves 14, 15 are opened and the pump 13 is activated. With the treatment head outlet 3.2 open via the bypass line 12, there exists a circulation of water vapor in the connection line, in the treatment head 3, and in the keg's interior 2.1. This means that during a holding time, turbulent mixing of water vapor in the keg's interior 2.1 promotes relative movement C between water vapor in the keg 2 and the keg's inner surfaces.

The relative movement C between the water vapor and the keg's inner surfaces significantly intensifies the water vapor's ability to treat the keg's inner surfaces as well as those of the keg fitting 4. This, in turn, accelerates the keg's heating and improves sterilization. Moreover, the relative movement C causes condensate that is on the walls to constantly be displaced, thus constantly exposing the wall to additional water vapor that has yet to be condensed. This means that the same section of the surface can repeatedly condense water vapor. This means greater heat transfer from the water vapor to the surface.

Condensate that, in the pressure phase, condenses in the keg interior 2.1 and collects in the lower region of the reusable keg 2 is transferred away via the part of the second line 8 that is in connection with the treatment-head outlet 3.2. From there, it makes its way to a separator 16.

Following the pressure phase, the third and fourth control-valves 14, 15 close. With the first line 5 still closed, an flushing gas flushes the keg's interior 2.1 to remove water vapor and condensate from the interior 2.1 and from the keg fitting 4 via the opened second line 8. A suitable flushing gas is the same gas used for pre-stressing. Suitable gases for these purposes are inert gases, such as carbon dioxide gas. After flushing, pre-stressing gas enters the keg's interior 2.1 to pre-stress it.

In alternative embodiments, relative movement between the water vapor and the keg's inner surface during the pressure phase arises from mechanically moving the keg 2 during this pressure phase. Movement can take the form jerking or shaking movement, rotation or slewing movement. This causes acceleration that will result in relative motion between the walls of the interior 2.1 and the water vapor. In some embodiments, such movement occurs about a mid-axis MA of the keg 2, which also happens to be the mid-axis of the treatment head 3 and of the riser pipe 4.1. In embodiments that achieve relative motion this way, the treatment position 1 includes a mechanical drive 17.

Having described the invention, and preferred embodiments thereof, what is claimed as new and secured by Letters Patent is:

1-14. (canceled)

15. A method comprising executing a pressurized phase of a vapor treatment process, wherein executing said pressurized phase comprises heating and sterilizing a surface to be sterilized, wherein said surface to be sterilized comprises interior walls of a metallic reusable keg having an interior and a keg fitting, wherein heating and sterilizing comprises connecting said interior to a water-vapor inlet through which pressurized water vapor enters said interior, interrupting said connection, thereby initiating a dwell time during which condensate forms on said surface to be sterilized, and during said dwell time, causing relative movement between said water vapor and said surface to be sterilized.

16. The method of claim 15, wherein causing relative movement comprises causing circulation of water vapor in said interior through a closed circuit in which water vapor is conveyed out of said interior and then back into said interior.

17. The method of claim 15, further comprising executing a pressureless phase of said vapor treatment prior to executing said pressurized phase, wherein executing said pressureless phase comprises causing water vapor to blow through said interior, wherein said water vapor enters said interior via said water-vapor inlet, and removing water vapor and condensate from said interior.

18. The method of claim 15, further comprising selecting said water vapor to be water vapor that is saturated and that is at a pressure above atmospheric pressure, wherein said water vapor is maintained at a temperature between 120° C. and 135° C.

19. The method of claim 15, further comprising, after having completed said pressure phase, using an inert gas to flush away water vapor and condensate from said interior.

20. The method of claim 19, further comprising introducing said inert gas into said interior via an inlet of a treatment head of said keg fitting and conveying away water vapor and condensate via an outlet of said treatment head.

21. The method of claim 15, further comprising ending said pressure phase after lapse of a predetermined time.

22. The method of claim 15, further comprising ending said pressure phase in response to a measurement indicating that a pressure in said interior has reached a threshold value.

23. The method of claim 15, further comprising sealing said keg against a treatment head such that said keg is sealed against said treatment head at least during said pressure phase.

24. The method of claim 23, further comprising delivering water vapor into said interior via an inlet of a treatment head and a riser pipe of said keg fitting and removing water vapor and condensate from said interior via an outlet of said treatment head.

25. The method of claim 15, further comprising jolting said keg.

26. The method of claim 15, further comprising slewing said keg.

27. The method of claim 15, further comprising shaking said keg.

28. The method of claim 15, further comprising rotating said keg.

29. The method of claim 15, further comprising causing a time-varying acceleration of said keg.

30. The method of claim 15, further comprising, prior to beginning said vapor treatment process, cleaning said interior.

31. The method of claim 15, further comprising executing a pressureless phase of said vapor treatment prior to executing said pressurized phase and selecting a dwell time of water vapor in said interior during said pressurized phase to be greater than the duration of said pressureless phase.

32. The method of claim 15, further comprising executing a pressureless phase of said vapor treatment prior to executing said pressurized phase and selecting a dwell time during said pressurized phase to be between sixty and ninety seconds.

33. An apparatus for carrying out vapor treatment of a surface to be treated, said surface to be treated comprising an interior wall of a reusable metallic keg having an interior, said surface further comprising a surface of a keg fitting, said apparatus comprising a treatment head, a first controlled-connection, a second controlled-connection, and means for causing relative movement between water vapor in said interior and said surface during a pressure phase of said vapor treatment, wherein said treatment head seals against said keg fitting when said keg is turned upside-down on said treatment head, wherein said first controlled connection connects a water-vapor inlet to a treatment-head inlet of said treatment head, and wherein said second controlled-connection conveys water vapor and condensate from said interior via an outlet of said treatment head.

34. The apparatus of claim 33, wherein said means for causing relative movement comprises a controlled bypass, a pump, and first and second connections for guiding vapor to and from said interior, wherein said pump is along said bypass, and wherein said bypass connects said first and second connections.

35. The apparatus of claim 33, wherein said means for causing relative movement comprises a mechanical drive for causing a time-varying acceleration of said keg.
